(19) United States
(12) Patent Application Publication
(10) Pub. No.: US 2008/0167317 A1
(43) Pub. Date: Jul. 10, 2008

(54) Combination Therapy

(76) Inventors: Anita Chugh, New Delhi (IN); Mohammad Salman, Princeton, NJ (US); Atul Tiwari, Haryana (IN); Gowri R. Shankar, Haryana (IN); Sandeep Sinha, New Delhi (IN)

Correspondence Address: RANBAXY INC., 600 COLLEGE ROAD EAST, SUITE 2100, PRINCETON, NJ, 08540, US

(21) Appl. No.: 10/593,939

(22) PCT Filed: Mar. 23, 2004
(86) PCT No.: PCT/IB2004/000866

§ 371 Date: Feb. 25, 2008

(30) Foreign Application Priority Data
Mar. 22, 2004 (IB) PCT/IB04/00842

(51) Int. Cl. A61K 31/496; A61P 13/00; A61K 31/18; A61K 31/403; A61K 31/47; A61K 31/235; A61K 31/473

(52) U.S. Cl. 514/253.01; 514/254.09; 514/602; 514/412; 514/307; 514/532; 514/284

(57) ABSTRACT

This invention relates to combination therapy for the treatment of lower urinary tract symptoms (LUTS) associated with or without benign prostatic hyperplasia (BPH). The combination therapy comprises of tailored 1 adrenoceptor antagonist, which is selective for 1a over 1b subtype but non-selective for 1a over 1d subtype, in combination with muscarinic receptor antagonist, preferably bladder selective antagonist and optionally included 5-reductase inhibitor for relief of LUTS in a mammal with or without BPH.

### FIELD OF THE INVENTION

This invention relates to combination therapy for the treatment of lower urinary tract symptoms (LUTS) associated with or without benign prostatic hyperplasia (BPH). The combination therapy comprises tailored α₁ adrenoceptor antagonists, which are selective for α₁ₐ over α_{1b} subtype but non-selective for α₁ₐ over α_{1d} subtype, in combination with muscarinic receptor antagonists, preferably bladder selective antagonists, and optionally included 5α-reductase inhibitor for relief of LUTS in a mammal, with or without BPH.

### BACKGROUND OF THE INVENTION

Benign prostatic hyperplasia also known as benign prostatic hypertrophy is highly prevalent in men beyond the age of 50 and increases in severity and incidence with increasing age. The incidence is 70% in 70 years and becomes nearly universal with advancing age with 90% incidence at the age of 80 years [Berry et al, J. Urol., 132:474-479, 1984].

Symptomatic BPH is thought to be due to bladder outflow obstruction and is usually suggestive of the lower urinary tract symptoms [Spealcman M. J., Eur. Urol. suppl., 40:21, 2001]. BPH is characterized by nodular enlargement of prostatic tissue and is associated with a variety of bothersome symptoms, which have a negative impact on quality of life. Lower urinary tract symptoms (LUTS) in men includes, but is not, restricted to a complex of obstructive (voiding) and irritative (storage or filling) symptoms, which include increased frequency, nocturia, poor urinary stream and hesitancy or delay in starting urinary flow. Chronic consequences of BPH can include hypertrophy of bladder smooth muscle, a decompensated bladder and increased incidence of urinary tract infections. Histologically, BPH is characterized by glandular (epithelial) and stromal (fibromuscular) hyperplasia with the latter being the dominant factor in the pathogenesis of clinically significant BPH [Shapiro et al, J. Urol., 147: 1293-1297, 1992].

Though the exact etiology of origin of these symptoms is not distinctly clear, two components, a static component and a dynamic component, clearly contribute to obstruction. Prostatic enlargement or hyperplasia of prostate gland physically impinges on the free flow of fluids through the male urethra and leads to varying degrees of bladder obstruction. This component has been referred as the static component [Caine M, J. Urol., 136:1-4, 1986]. Increased adrenergic innervation to prostate leads to an increased adrenergic tone of the bladder neck or urethra and is referred to as dynamic component. The irritative symptoms have been closely associated with bladder dysfunction, which was believed to be a consequence of bladder outlet obstruction [Anderson K E, Brit. J. Urol, 85 Suppl: 12-18, 2000].

Standard treatments for BPH involve surgical or pharmacological intervention. Surgical intervention involves removal of the prostate via radical prostectomy or removing the prostate adenoma via transurethral resection of the prostate. These invasive surgical procedures have limited utility because of the morbidity associated with operative procedures as well as the persistence and recurrence of obstructive and irritative symptoms. Surgical procedures are, therefore, not recommended for patients exhibiting mild to moderate symptoms.

Presently, pharmacological interventions in the treatment of BPH can be distinctly categorized into two main categories: alpha-1 adrenergic receptor antagonists and 5-alpha reductase inhibitors. 5-alpha reductase inhibitors such as finasteride and dutasteride reduce the size of prostate [Wilde et al, Drugs, 57:557-581, 1999], thereby alleviating the static component of bladder outlet obstruction. The lesser efficacy associated with these inhibitors is mechanism-based, in that 5-alpha reductase inhibitors decrease the size of prostate by reducing the amount of epithelial tissue without affecting the smooth muscle and the dynamic component of bladder outlet obstruction.

Other pharmacological therapy involves the administration of subtype non-selective alpha-1 adrenoceptor antagonists. These agents relax prostatic-urethral smooth muscle by blocking the alpha-1 mediated effects on endogenous tone hence affecting the dynamic component of bladder outlet obstruction and relieving obstructive symptoms [Chapple, Brit J. Urol., 1:47-55, 1995, Kawabe and Niijima, Urol Int., 42:280-284, 1987, Lepor et al, J. Urol., 148:1467-1474, 1992, Reuther and Aagard, Urol. Int., 39:312-313, 1984, Serels and Stein, Neurourol. Urodyn., 17:31-36, 1998]. In addition these alpha-1 adrenoceptor antagonists have also been found to relieve the irritative bladder symptoms associated with BPH.

Alpha adrenoceptors are members of a larger G protein-coupled adrenergic receptors family, which mediate the actions of endogenous catecholamines norepinephrine and epinephrine resulting in smooth muscle contraction. cDNA's encoding three distinct alpha-1 adrenoceptor subtype (alpha-1a, alpha-1b and alpha-1d) and three distinct alpha-2 adrenoceptor subtypes (alpha-2a, alpha-2b and alpha-2c) have been cloned, expressed stably in cells and resultant protein characterized pharmacologically, [Schwinn et al, J. Pharmacol. Exper. Ther., 272:134-142, 1995, Hieble et al Pharinacol. Rev., 47:267-70, 1995].

Human lower urinary tract contains both alpha-1 and alpha-2 adrenoceptors, with the latter predominating the formner [Goepel et al, Urol. Res., 25:199-206, 1997]. However the prostatic smooth muscle contraction is mediated predominantly, if not exclusively by alpha-1-adrenoceptors [Hieble et al, Eur. Pharmacol., 107:111-117, 1985, Chappel et al, Br. J. Urol., 63:487-496, 1989].

Alpha-1 adrenoceptors predominate in prostate and bladder trigone, [Price et al J. Urol., 150:546-551, 1993], and have been shown to be functionally important in mediating smooth muscle contraction [Forray et al, Mol. Pharmacol., 45:703-708, 1994, Lepor et al J. Pharmacol. Exper. Ther., 270:722-727, 1994]. In addition to the three cloned alpha-1 adrenoceptor subtypes, which have high affinity for the prazosin a fourth type of α₁AR with low affinity for prazosin (α_{1L}) has been postulated [Muramatsu et al, Br. J. Urol., 74: 572-578 (1994)]. However, there is evidence to suggest that it may represent functional phenotype of the alpha-1AR [Daniels D. V., Eur. J. Pharmacol., 370:37-43, 1990].

The non-subtype selective alpha-1 adrenoceptor antagonists, such as prazosin, terazosin, doxazosin and alfuzosin are accompanied by side effects such as postural hypotension, dizziness and syncope. These side effects are attributed to the affinity towards non-selective alpha-1 adrenoceptor subtypes in the vasculature [J. Androl., 18: 345-355, 1991]. Therefore, in an attempt to develop alpha-1 adrenoceptor antagonist with minimal cardiovascular effect, the concept of developing α_{1A} subtype selective antagonists with minimal affinity for α_{1B} and α_{1D} subtype in BPH was proposed which is extensively covered in method of use by Synaptic and reviewed in U.S. Pat. Nos. 5,403,847; 5,578,611; 5,780,485; 5,990,128; and 6,015,819.

Development of several α_{1A} subtype selective compounds with minimal affinity for α_{1B} and/or α_{1D} adrenoceptor has been reported. The selectivity at α_{1A} to α_{1B} adrenoceptor is important as such antagonists cause significantly smaller blood pressure alterations and fewer cases of orthostatic hypertension, as compared to nonselective α₁ adrenoceptor antagonists [Michael M C, Eur. Urol. Suppl., 5-13, 2002].

Recent studies, however suggest that the relief of bladder outlet obstruction only partly explain involvement of lower urinary tract with these agents. There is poor correlation in BPH patients between obstructive (voiding) and irritative (storage) sypptoms and urine flow rates at base line. The irritative symptoms can persist despite the relief of bladder outlet obstruction [Hieble and Ruffolo, J. Exp. Opin. Invest. Drug, 6:367-387, 1997].

In recent clinical studies with experimental antagonists with high affinity for a α_{1A} adrenoceptors and particularly devoid of α_{1D} activity have demonstrated enhancements in the urine flow rates without any improvement on irritative lower urinary tract symptoms [Blue et al, J. Urol., 167(Suppl):265, 2002].

Irritative symptoms such as urgency and frequency traditionally associated with BPH, are also observed in lower urinary tract in women suffering from detrusor instability suggesting that these symptoms are caused by similar mechanisms or are amenable to a single form of therapy [Staskin D R et al, Urology, 60 (Suppl 5A): 1-6, 2002].

The two main functions of the urinary bladder are to store urine and to empty it, by involving a complex pattern of nerve signalling. Disturbances in the normal control of the bladder reflexes may lead to an “overactive bladder”, clinically characterized by symptoms of urgency, frequency, nocturia and urge incontinence. Bladder excitability is under the control of parasympathetic nervous system and releases the neurotransmitter acetylcholine. Acetylcholine acts on protein recognition sites in bladder known as muscarinic receptors.

Muscarinic receptors are G-protein coupled receptors, encoded by five distinct genes [Caulfield and Birdsall, Pharmacol. Rev., 50:279-290, 1998]. These genes characterize five distinct molecular and pharmacological subtypes namely M1, M2, M3, M4 & M5. Normal human bladder contraction is mediated mainly through stimulation of muscarinic receptors in detrusor muscle by the endogenous ligand, acetycholine. The muscarinic receptors found in human detrusor are of M2 and M3 subtypes [Hedge and Eglen, Life Sci., 64:419-428, 1999, Fetscher et al, Brit. Jr. Pharmacol., 136:641-644, 2002]. M2 receptors predominate in number over M3 subtype but it is M3 receptors, which are mainly responsible for the normal micturition contraction [Yamanishi et al, World J. Urol., 19:299-306, 2001]. Muscarinic receptors are involved in both normal and disturbed bladder contraction, and therefore the most common drug treatment of overactive bladder is muscarinic receptor antagonists also referred as antimuscarinic drugs. Antimuscarinics block more or less selectively muscarinic receptors on the bladder smooth muscle (detrusor), which are stimulated by acetylcholine. Thereby they decrease the ability of bladder to contract. Antimuscarinic drugs act mainly during the storage phase, increase the bladder capacity and decrease the urge.

In the patients of outflow obstruction, as in BPH, muscarinic receptor antagonists have generally been contraindicated for symptomatic relief because of the possible risk of acute urinary retention [Sullvian et al, Eur. Urol., 36 (Suppl 1):89-95, 1999]. A number of reports of urinary outflow obstruction induced in patients who were given ipratropium by aerosol for respiratory conditions have also been recorded [Lozewicz S, Postgrad Med. J., 65:260-261, 1989]. These patients were found to have enlarged prostate gland.

However recently a combination treatment of an alpha blocker (tamsulosin) plus an anticholinergic (tolterodine) has been reported to improve the quality of life in patients with bladder obstruction and concomitant detrusor instability with no acute urinary retention [Athanasopoulos et al, J. Urol., 169:2253-2256, 2003]. In another evidence, administration of tolterodine, a antimuscarinic drug in men with bladder outlet obstruction and symptomatic detrusor overactivity was reported not to be associated with any safety concern [Abrams P et al., Eur. Urol., 1:132, 2002 (abstract 520)].

A combination of a dyphylline-type compound with α AR antagonist and/or 5α-reductase inhibitor for the treatment of BPH has been disclosed in U.S. Pat. No. 6,423,719. WO 99/57131 discloses a method of identifying α_{1d} AR antagonists that can be used to treat irritative symptoms of BPH. A combination of α₁ₐ AR antagonist with 5α-reductase inhibitor for the treatment of BPH has been disclosed in U.S. Pat. No. 6,376,503. A method of treating LUTS and pharmaceutical composition comprising a muscarinic receptor antagonist and at least one other active ingredient selected from a 5α-reductase inhibitor and an α AR antagonist have been disclosed in WO 01/21167. Pharmaceutical combinations comprising α AR antagonist and a muscarinic receptor antagonist for the treatments of LUTS associated with BPH in men are disclosed in US Patent Application No. 2001/0044438.

### SUMMARY OF THE INVENTION

A combination of α_{1A}/α_{1D} non-selective antagonist and bladder selective antagonists can offer advantages of relieving LUTS (both obstructive and irritative symptoms) more effectively in patients with BPH, with minimal side effects such as fall in blood pressure and the antimuscarinic associated side effects such as dry mouth and other undesirable side effects. The present combination is proposed to be safe and effective in BPH patients to alleviate the lower urinary tract symptoms with bladder outlet obstruction and concomitant detrusor instability. This combination will also offer advantages for alleviation of obstructive lower urinary tract symptoms in women and treatment of lower urinary tract symptoms in men in absence of BPH.

Accordingly, herein is provided a combination of a tailored α₁ adrenoceptor antagonist, which is selective for α₁ₐ over α_{1b} subtype but non-selective for α₁ₐ over α_{1d} subtype, with a muscarinic receptor antagonist, for example a bladder-selective antagonist and optionally included 5α-reductase inhibitors, for use as a medicament for the treatment of LUTS in mammal associated with or without BPH.

The product or medicament provided herein can be a combined preparation of a first pharmaceutically acceptable composition containing a tailored α₁ AR antagonist, which is selective for α₁ₐ over α_{1b} subtype but non-selective for α₁ₐ over α_{1d} subtype, a second composition containing a muscarinic receptor antagonist, for example, a bladder-selective antagonist and optionally included third pharmaceutically acceptable composition containing 5α-reductase inhibitor. The components of such a combined preparation may be administered simultaneously, separately or sequentially.

Also provided herein is a pharmaceutical composition comprising a tailored α₁ AR antagonist, which is selective for α₁ₐ over α_{1b} subtype but non-selective for α₁ₐ over α_{1d} subtype, a muscarinic receptor antagonist, for example, a bladder-selective antagonist and optionally included 5α-reductase inhibitor and a pharmaceutically acceptable carrier for the treatment of LUTS associated with or without BPH.

Also provided herein is a method for the treatment of LUTS associated with or without BPH in a mammal comprising administering to mammal in need thereof an effective amount of a tailored α₁ AR antagonist, which is selective for α₁ₐ over α_{1b} subtype but non-selective for α₁ₐ over α_{1d} subtype, in combination with a muscarinic receptor antagonist, for example, a bladder-selective antagonist and optionally included 5α-reductase inhibitor. The combination may be administered simultaneously, separately or sequentially.

### DETAILED DESCRIPTION OF THE INVENTION

As used herein the term “tailored α₁ adrenoceptor antagonists” refer to those agents, which are more than about 20, or more than about 10-fold selective for α₁ₐ as compared to α_{1b} subtype and are less than about 20, or less than about 10 fold selective for α₁ₐ over α_{1d} subtype AR antagonist in receptor binding and in vitro functional assay.

The tailored α₁ AR antagonists can be selected from, for example:
1-{3-[4-(2-methoxyphenyl) piperazin-1-yl]-propyl}-piperidine-2,6-dione,
2-[3-{4-(2-isopropoxyphenyl)piperazin-1-yl}propyl]-3a,4,7,7a-tetrahydro-1H-isoindole-1,3(2H)-dione,
5-[2-[[2-(2-ethoxyphenoxy)ethyl]amino]propyl]-2-hydroxybenzenesulfonamide, or their pharmaceutically acceptable salts, pharmaceutically acceptable solvates, enantiomers, racemate, polymorphs, N-oxides or metabolites.

In one particular embodiment, the tailored α₁ AR antagonists can be selected from, for example:
1-{3-[4-(2-methoxyphenyl) piperazin-1-yl]-propyl}-pipeiidine-2,6-dione hydrochloride salt,
2-[3-{4-(2-isopropoxyphenyl)piperazin-1-yl}propyl]-3a,4,7,7a-tetrahydro-1H-isoindole-1,3(2H)-dione hydrochloride salt and
5-[2-[[2-(2-ethoxyphenoxy)ethyl]amino]propyl]-2-hydroxybenzenesulfonamide hydrochloride salt.

As used herein the term “bladder selective antagonists” refer to those agents, which exhibit greater potency in inhibiting the carbachol-induced response on the bladder than the carbachol-evoked salivation when evaluated simultaneously in in vivo model in rabbit or dog.

The bladder-selective antagonists can be selected from, for example:
(1α,5α,6α)-N-[3-benzyl-3-azabicyclo[3.1.0]hexyl-6-(aminomethyl)-yl]-2-hydroxy-2-cyclopentyl-2-phenyl acetamide, or
(1α,5α,6α)-[3-benzyl-3-azabicyclo[3.1.0}hexyl-6-(methyl)-yl]-2-hydroxy-2,2-diphenyl acetate,
(1α,5α,6α)-[3-benzyl-3-azabicyclo[3.1.0]hexyl-6-(methyl)-yl]-2-hydroxy-2-cyclohexyl-2-phenyl acetate,
(1α,5α,6α)-[3-benzyl-3-azabicyclo[3.1.0]-hexyl-6-(methyl)-yl]-2-hydroxy-2-cyclopentyl-2-phenyl acetate,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamnide,
N-[(1α,5α,6α)-3-azabicyclo[3.1.0]-hex-6-ylmethyl]-2-isopropyl-2-hydroxy-2-phenyl acetamide,
N-{[(1α,5α,6α)-3-chloro-3-azabicyclo[3.1.0]hex-6ylmethyl]}-2-cyclopentyl-2-hydroxy-2-phenyl acetamide,
(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide,
(1α,5α,6α)-N-[3-benzyl-3-azabicyclo[3.1.0]hexyl-6-(aminomethyl)-yl]-2-hydroxy-2-cyclohexyl-2-phenyl acetamide,
(1α,5α,6α)-N-[3-(1-phenylethyl)-3-azabicyclo[3.1.0]hexyl-6-(amino)-yl]-2-hydroxy-2-cyclopentyl-2-phenyl acetamide,
(1α,5α,6α)-N-[3-benzyl-3-azabicyclo[3.1.0]hexyl-6-(aminomethyl)-yl]-2-hydroxy-2,2-diphenyl acetamide,
3-azabicyclo[3.1.0]hex-3-yl]but-2-ynyl-2-cyclopentyl-2-hydroxyphenyl acetate,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide,
(1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide, or
   their pharmaceutically acceptable salts, pharmaceutically acceptable solvates, enantiomers, diastereomers, polymorphs, N-oxide or metabolites.

In another particular embodiment, the bladder-selective antagonists can be selected from, for example:
(1α,5α,6α)-N-[3-benzyl-3-azabicyclo[3.1.0]hexyl-6-(aminomethyl)-yl]-2-hydroxy-2-cyclopentyl-2-phenyl acetamide L-(+)-tartrate salt,
(1α,5α,6α)-[3-benzyl-3-azabicyclo[3.1.0}hexyl-6-(methyl)-yl]-2-hydroxy-2,2-diphenyl acetate L(+)-tartrate salt,
(1α,5α,6α)-[3-benzyl-3-azabicyclo[3.1.0]hexyl-6-(methyl)-yl]-2-hydroxy-2-cyclohexyl-2-phenyl acetate L(+)-tartrate salt,
(1α,5α,6α)-[3-benzyl-3-azabicyclo[3.1.0]-hexyl-6-(methyl)-yl]-2-hydroxy-2-cyclopentyl-2-phenyl acetate L(+)-tartrate salt,
(2R)-(+)-(1α,5α,6α)-N-[3-benzyl-3-azabicyclo[3.1.0]hexyl-6-(aminomethyl)-yl]-2-hydroxy-2-cyclopentyl-2-phenyl acetamide L(+)-tartrate salt,
(2R,2S) (1α,5α,6α)-N-[3-azabicyclo[3.1.0]hexyl-6-(aminomethyl)-yl]-2-hydroxy-2-cyclopentyl-2-phenyl acetamide hydrochloride salt,
(2R)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2S)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2R,2S) (1α,5α,6α)-N-[3-azabicyclo[3.1.0]hexyl-6-(aminomethyl)-yl]-2-hydroxy-2-(3,3-difluorocyclopentyl)-2-phenyl acetamide tartrate salt,
(2R,2S) (1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamide tartrate salt,
(2R,2S)-N-[(1α,5α,6α)-3-azabicyclo[3.1.0]-hex-6-ylmethyl]-2-isopropyl-2-hydroxy-2-phenyl acetamide hydrochloride salt,
N-{[(1α,5α,6α)-3-chloro-3-azabicyclo[3.1.0]hex-6ylmethyl]}-2-cyclopentyl-2-hydroxy-2-phenyl acetamide hydrochloride salt,
(2R)-(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide tartrate salt,
(2R)-(1α,5α,6α)-N-[3-benzyl-3-azabicyclo[3.1.0]hexyl-6-(aminomethyl)-yl]-2-[(1S or 1R)-3,3-difluorocyclopentyl]-2-hydroxy-2-phenyl acetamide tartrate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide succinate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide tartrate salt,
(2R,2S)-(1α,5α,6α)-N-[3-(1-phenylethyl)-3-azabicyclo[3.1.0]hexyl-6-(amino)-yl]-2-hydroxy-2-cyclopentyl-2-phenyl acetamide tartrate salt,
(2R)-(1α,5α,6α)-N-[3-benzyl-3-azabicyclo[3.1.0]hexyl-6-(aminomethyl)-yl]-2-hydroxy-2-cyclopentyl-2-phenyl acetamide tartrate salt,
(1α,5α,6α)-N-[3-benzyl-3-azabicyclo[3.1.0]hexyl-6-(aminomethyl)-yl]-2-hydroxy-2,2-diphenyl acetamide tartrate salt,
2R(+),4[(1R,5S)-3-azabicyclo[3.1.0]hex-3-yl]but-2-ynyl-2-cyclopentyl-2-hydroxyphenyl acetate hydrochloride,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide L(+) tartrate salt,
(2R) (1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(2R)-(1α,5α,6α)-6-N-(3-azabicyclo[3.1.0]hexyl-3-(4-methyl-3-pentenyl))-2-cyclopentyl-2-hydroxy-2-phenyl acetamide succinate salt,
(2R)-(1α,5α,6α)-6-N-(3-azabicyclo[3.1.0]hexyl-3-(4-methyl-3-pentenyl))-2-cyclopentyl-2-hydroxy-2-phenyl acetamide L(+) tartrate salt,
(1S)-(3R)-1-azabicyclo[2,2,2]oct-3-yl-3,4-dihydro-1-phenyl-2(1H)-isoquinolinecarboxylate,
(1S)-(3R)-1-azabicyclo[2,2,2]oct-3-yl-3,4-dihydro-1-phenyl-2(1H)-isoquinolinecarboxylate succinate salt,
2-methyl propanoic acid 2-[(1R)-3-[bis(1-methylethyl)amino]-1-phenylpropyl]-4-(hydroxymethyl)phenyl ester and
2-methyl propanoic acid 2-[(1R)-3-[bis(1-methylethyl)amino]-1-phenylpropyl]-4-(hydroxymethyl)phenyl ester with (2E)-2-butenedioate.

As used herein the term “5α-reductase” refers to enzymes which catalyze the conversion of testosterone (T) to dihydrotestosterone (DHT) in androgen-responsive tissues such as prostate, seminal vesicles, epididymis and skin. Two isoforms of 5α-reductase have been described-Type 1 and Type 2 (Ranjan et al., Life Sci., 71:115-126, 2002). Type 1 5α-reductase is the predominant enzyme in extraprostatic tissues such as skin and liver whereas Type 2 enzyme is predominantly expressed in the prostate. The two enzymes differ in their catalytic and biochemical properties such as Kₘ, pH optimum etc., (Andriole and Kirby, Eur. Urol., 44:82-88, 2003).

The 5α-reductase inhibitor may be widely chosen from among those already known to the prior art or subsequently discovered and/or hereafter discovered and/or hereafter developed. Compounds that are inhibitors of testosterone 5a-reductase inhibitor have been disclosed in U.S. Pat. No. 5,595,985, U.S. Pat. No. 4,377,584, U.S. Pat. No. 4,760,071, U.S. Pat. No. 5,017,568, U.S. Pat. No. 5,155,107, U.S. Pat. No. 5,565,467, EP 0572165, WO 93/23420, EP 0572166, WO 93/23050, WO 93/23038, WO 93/23048, WO 93/23041, WO 93/23040, WO 93/23039, WO93/23376, WO 93/23419, and WO 93/23051, and these patents are incorporated by reference herein in their entirety. Compounds may be inhibitor of a type-1 or type-2 testosterone 5α-reductase isoenzymes or both a type-1 and type-2 or a dual type-1 and type-2. These compounds can be selected from finasteride, dutasteride, epristeride and turosteride, for example.

Also provided herein are pharmaceutically acceptable salt of compounds disclosed herein. The pharmaceutically acceptable salts can include, for example, alkali metal salts and addition salts of acids or bases. Suitable pharmaceutically acceptable acid addition salts may be prepared from an inorganic acid or from an organic acid. Example of such inorganic acids include, but are not limited to, hydrochloric, hydrobromic, hydroiodic, nitrous (nitrite salt), nitric (nitrate salt), carbonic, sulfuric, phosphoric acid and like. Appropriate organic acids include, but are not limited to, aliphatic, cycloaliphatic, aromatic, heterocyclic, carboxylic and sulfonic classes of organic acids, such as, for example, formic, acetic, propionic, succenic, glycolic, gluconic, lactic, malic, tartaric, dihydroxytartaric acid, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfoxic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galacturonic acid and the like. Suitable pharmaceutically acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, and procaine and the like. The salt forms can generally differ from the base forms of the compounds described herein in certain physical properties such as solubility in polar solvent.

Prodrugs of these agents are also included. In general, such prodrugs will be functional derivatives of these compounds, which are readily convertible in vivo into the required compound. Conventional procedure for the selection and preparation of suitable prodrug derivatives are described, for example, in “Design of Prodrugs”, ed. H Bundgaard and, Elsevier, 1985. The present invention also includes metabolites, which become active upon introduction into biological systems. Where the compounds according to the invention have at least one chiral center, they may accordingly exist as enantiomers. Where the compounds according to invention possess two or more chiral centers, they may additionally exist as diastereomers. It is to be understood that all such isomers and racemic mixtures therefore are encompassed within the scope of the present invention. Furthermore, some of the crystalline forms for compounds described herein may exist as polymorphs and as such are intended to be included in the present invention. In addition, some of the compounds described herein may form solvates with water (i.e., hydrates) or common organic solvents. Such solvates are also encompassed within the scope of this invention.

In accordance with one aspect, there is provided a product or medicament comprising a pharmaceutically acceptable composition containing a therapeutically effective amount of a tailored α₁ AR antagonist, which is selective for α₁ₐ over α_{1b} subtype but non-selective for α₁ₐ over α_{1d} subtype, a second pharmaceutically acceptable composition containing therapeutically effective amount of a muscarinic receptor antagonist, for example, a bladder-selective antagonist and optionally included therapeutically effective amount of 5α-reductase inhibitor as a combined preparation for simultaneous, separate or sequential for the treatment of LUTS with or without BPH. LUTS may include, for example, obstructive symptoms such as hesitancy, poor stream, prolong urination, and feelings of incomplete emptying, and irritative symptoms such as frequency, urgency, nocturia and bladder contractions, in a mammal in need thereof. The term “therapeutically effective amount”, as used herein means that amount of active compound that elicits the biological or medicinal response in a mammal which includes at least partial alleviation of the symptoms of the disease being treated.

In accordance with a second aspect, there is provided single composition containing a therapeutically effective amount of a tailored α₁ AR antagonist, which is selective for α₁ₐ over α_{1b} subtype but non-selective for α₁ₐ over α_{1d} subtype, a therapeutically effective amount of a muscarinic receptor antagonist, for example, a bladder-selective antagonist and optionally a therapeutically effective amount of 5α-reductase inhibitor for the treatment of LUTS with or without BPH. LUTS may include, for example, obstructive symptoms, such as hesitancy, poor stream, prolonged urination and feelings of incomplete emptying, and irritative symptoms such as frequency, urgency, nocturia, and unstable bladder contractions.

In accordance with a third aspect, there is provided a pharmaceutical composition containing a tailored α₁ AR antagonist, which is selective for α₁ₐ over α_{1b} subtype but non-selective for α₁ₐ over α_{1d} subtype, a muscarinic receptor antagonist, for example, a bladder-selective antagonist and optionally 5-alpha reductase inhibitor in combination with pharmaceutically acceptable carriers, diluents or excepients.

The compositions disclosed herein include both those containing only one component and those containing a tailored α₁ AR antagonist, which is selective for α₁ₐ over α_{1b} subtype but non-selective for α₁ₐ over α_{1d} subtype, a muscarinic receptor antagonist, for example, a bladder-selective antagonist and optionally included 5α-reductase inhibitor and which, may be suitable for oral, parenteral, topical, transdermal, cholonic or intravaginal administration. The composition may be formulated to provide immediate or sustained release of the therapeutic agents. The agents described herein can be administered alone but will generally be administered as an admixture with a suitable “pharmaceutically acceptable carrier”. The term “pharmaceutically acceptable carrier” is intended to include non-toxic, inert solid, semi-solid or liquid filter, diluent, encapsulating material or formulation auxiliary of any type.

Solid form preparations for oral administration may include capsules, tablets, pills, powders, granules and suppositories. For solid-form preparations, the active compound is mixed with at least one inert, pharmaceutically acceptable excipient or carrier such as sodium citrate, dicalcium phosphate and/or a filter an extender such as starch, lactose, sucrose, glucose, mannitol and silicic acid; binders such as carboxymethyl cellulose, alginates, gelatins, polyvinylpyrrolidinone, sucrose, acacia; disintegrating agents such as agar-agar, calcium carbonate, potato starch, aliginic acid, certain silicates and sodium carbonate; absorption accelators such as quaternary ammonium compounds; wetting agents such as cetyl alcohol, glycerol, monostearate; adsorbents such as kaolin; lubricants such as talc, calcium stearate, magnesium stearate, solid polyethyleneglycol, sodium lauryl sulphate and mixtures thereof.

In case of capsules, tablets, or pills, the dosage form may also comprise buffering agents. The solid preparation of tablets, capsules, pills, granules can be prepared with coatings and shells, such as enteric coating and other coatings well known in the pharmaceutical formulating art.

Liquid-form preparations for oral administration can include pharmaceutically acceptable emulsions, solution, suspensions, syrups and elixirs. For liquid-form preparations, the active compound can be mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as cottonseed, groundnut, corn, germ, olive, castor and sesame oil), glycerol and fatty acid ester of sorbitan and mixtures thereof.

Besides inert diluents, oral compositions can also include adjuvants such as wetting agents, emulsifying agents, suspending agents, sweetening agents, flavoring agents and perfuming agents.

Injectible preparations such as sterile injections, aqueous or oleaginous suspensions may be formulated according to the art using suitable dispersing or wetting and suspending agents. Among the acceptable vehicles and solvents that may be employed are water, Ringers solution and isotonic sodium chloride.

Dosage forms for topical or transdermal administration can include ointments, pastes, creams, lotions, gel, powders, solutions, sprays, inhalants or patches. The active compound can be admixed under sterile conditions with a pharmaceutically acceptable canier and preservatives or buffers as may be required.

The pharmaceutical preparations can be in unit dosage form. In such form, the preparation can be subdivided into unit doses containing appropriate quantities of the active component.

The formulations as described herein may be formulated so as to provide quick, sustained, or delayed release of the active ingredient after administration to the patient by employing procedures well known to the art. The compositions may be administered as a depot formulation that permits sustained release, limits access to general circulation, and increases the prostate and/or bladder-specific localization of the composition. Such formulations may be provided as slow release implants, be microencapsulated, or attached to biodegradable polymers or prostate-specific immunoglobulins. The compound can be administered in a sustained release formulation as a tablet or capsule. A sustained release formulation is a preparation that releases the active component over a desired period of time after administration. A sustained release formulation is prepared by applying a biodegradable, bioerodible or bioabsorbable polymeric formulation that is compatible on the surface of the active component. Examples of sustained release formulation include, but are not limited to, hydroxypropylmethylcellulose (HPMC), hydrogenated vegetable oil (HVO), ethyl cellulose, polyvinylpyrrolidione, pyran copolymer, polyhydroxypropylmethacryl—amidephenol, polyhydroxy—ethylaspartamidephenol, or polyethyleneoxidepolylysin substituted with palmitoyl residues, polylactic acid, polyepsilon caprolactone, polyhydroxy butyric acid, polyorthoesters, polyacetals, polydihydro-pyrans, and polycyano acrylates.

The term “biodegradable” means that the polymeric formulation degrades over time by the action of enzymes, by hydrolytic action and/or by other mechanisms in the human body. By “bioerodible” it is meant that the polymeric formulation erodes or degrades over time due, at least in part, to contact with substances found in the surrounding tissue fluids or cellular action. By “bioabsorbable”, it is meant that the polymeric formulation is broken down and absorbed within the body of a mammal, for example, by a cell or tissue. “Biocompatible” means that the polymeric formulation does not cause substantial tissue irritation or necrosis.

The compounds described herein can also be administered in the form of liposome delivery systems, for example, small unilamellar vesicles, large unilamellar vesicles and multilamellar vesicles. Liposomes can be formed from a variety of phospholipids, for example, cholesterol, stearylamine or phosphatidylcholines.

Herein are also disclosed aqueous parenteral compositions, containing a therapeutically effective amount of a tailored α₁ AR antagonist, which is selective for α₁ₐ over α_{1b} subtype but non-selective for α₁ₐ over α_{1d} subtype, a muscarinic receptor antagonist, for example, a bladder-selective antagonist and optionally included 5-alpha reductase inhibitor. The invention also provides a method of delivery such that direct intraprostatic injection of a therapeutically effective amount of disclosed compositions results in the relief of obstructive symptoms associated with benign prostatic hyperplasia.

Also disclosed herein is a method of treating LUTS with or without BPH. LUTS may include, for example, obstructive symptoms such as hesitancy, poor stream, prolonged urination, and feelings of incomplete emptying, and irritative symptoms such as frequency, urgency, nocturia and bladder contractions caused by BPH, comprising the administration of a therapeutically effective amount of a tailored α₁ AR antagonist, which is selective for α₁ₐ over α_{1b} subtype but non-selective for α₁ₐ over α_{1d} subtype, therapeutically effective amount of a muscarinic receptor antagonist, for example, a bladder-selective antagonist and optionally included 5α-reductase inhibitor to mammal in need thereof. The combined preparation can be administered simultaneously, separately or sequentially. As used herein the term “combined preparation” refers to a product or medicament comprises a container (packaging device well known to one ordinary skilled in the art) containing separate pharmaceutical compositions [same or different dosage forms, for example, oral (such as capsules, tablets, pills, powder, granules, suppository, emulsions, solution, suspensions, syrups or elixirs), injectible, topical or transdermal (such as ointments, pastes, creams, lotions, gel, powders, solutions, spray, inhalants or patches) of tailored ai adrenoceptor antagonist, bladder selective antagonist and optionally 5α-reductase inhibitor.

Also disclosed herein is a method for the treatment of LUTS with or without BPH, comprising administering a single dosage form containing a therapeutically effective amount of a tailored α₁ AR antagonist, which is selective for α₁ₐ over α_{1b} subtype but non-selective for α₁ₐ over α_{1d} subtype, therapeutically effective amount of a muscarinic receptor antagonist, for example, a bladder-selective antagonist, and optionally included 5α-reductase inhibitor to a mammal in need thereof.

The suitability of a tailored α₁ AR antagonist in this invention can be determined using for example, the assay methods those disclosed in J. Auton. Pharmacol., 16:21, 1996.

The suitability of a muscarinic receptor antagonist, for example, a bladder selective antagonist in this invention can be determined using for example the assay methods those disclosed in Life Sci., 64:2351, 1999 and J. Med. Chem., 42:1999, 1999.

The pharmaceutical compositions as described herein can be administered together combined in a single dosage form or they can be administered separately, simultaneously or sequentially, each in its dosage form but as part of the same therapeutic treatment program or regimen. Separate administration of each compound, at different times and by different routes, will sometimes be recommended.

Other pharmaceutical components may also optionally be included as part of the combination for the treatment of BPH and LUTS associated with or without BPH.

We claim:

1. A pharmaceutical composition comprising a tailored α₁-adrenoceptor antagonist, a bladder-selective antagonist and optionally included 5α-reductase inhibitor, optionally together with pharmaceutically acceptable carriers, excipients or diluents.

2. The pharmaceutical composition according to claim 1 wherein the tailored α₁ AR antagonist is selective for α₁ₐ over aIb subtype but non-selective for α₁ₐ over α_{1d} subtype.

3. The pharmaceutical composition according to claim 1 wherein the tailored α₁ AR antagonist is more than about 10 fold selective for ala over α_{1b} subtype and is less than about 10 fold selective for α₁ₐ over α_{1d} subtype in receptor binding and in vitro functional assay.

4. The pharmaceutical composition according to claim 3 wherein the tailored α₁ adrenoceptor antagonist is selected from:
1-{3-[4-(2-methoxyphenyl) piperazin-1-yl]-propyl}-piperidine-2,6-dione,
2-[3-{4-(2-isopropoxyphenyl)piperazin-1-yl}propyl]-3a,4,7,7a-tetrahydro-1H-isoindole-1,3(2H)-dione,
5-[2-[[2-(2-ethoxyphenoxy)ethyl]amino]propyl]-2-hydroxybenzenesulfonamide,
and their pharmaceutically acceptable salts, pharmaceutically acceptable solvates, enantiomer, racemate, polymorphs, N-oxides or metabolites.

5. The pharmaceutical composition according to claim 3 wherein the tailored α₁ adrenoceptor antagonist is selected from:
1-{3-[4-(2-methoxyphenyl) piperazin-1-yl]-propyl}-pipeiidine-2,6-dione hydrochloride salt,
2-[3-{4-(2-isopropoxyphenyl)piperazin-1-yl}propyl]-3a,4,7,7a-tetrahydro-1H-isoindole-1,3(2H)-dione hydrochloride salt and
5-[2-[[2-(2-ethoxyphenoxy)ethyl]amino]propyl]-2-hydroxybenzenesulfonamide hydrochloride salt,

6. The pharmaceutical composition according to claim 1, wherein the bladder selective antagonist is an agent which exhibits greater potency in inhibiting the carbachol-induced response on the bladder than the carbachol-evoked salivation when evaluated simultaneously in in vivo model in rabbit or dog.

7. The pharmaceutical composition according to claim 6 wherein the bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide, or
(1α,5α,6α)-[3-benzyl-3-azabicyclo[3.1.0}hexyl-6-(methyl)-yl]-2-hydroxy-2,2-diphenyl acetate,
(1α,5α,6α)-[3-benzyl-3-azabicyclo[3.1.0]hexyl-6-(methyl)-yl]-2-hydroxy-2-cyclohexyl-2-phenyl acetate,
(1α,5α,6α)-[3-benzyl-3-azabicyclo[3.1.0]-hexyl-6-(methyl)-yl]-2-hydroxy-2-cyclopentyl-2-phenyl acetate,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamnide,
N-[(1α,5α,6α)-3-azabicyclo[3.1.0]-hex-6-ylmethyl]-2-isopropyl-2-hydroxy-2-phenyl acetamide,
N-{[(1α,5α,6α)-3-chloro-3-azabicyclo[3.1.0]hex-6ylmethyl]}-2-cyclopentyl-2-hydroxy-2-phenyl acetamide,
(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide,
(1α,5α,6α)-N-[3-benzyl-3-azabicyclo[3.1.0]hexyl-6-(aminomethyl)-yl]-2-hydroxy-2-cyclohexyl-2-phenyl acetamide,
(1α,5α,6α)-N-[3-(1-phenylethyl)-3-azabicyclo[3.1.0]hexyl-6-(amino)-yl]-2-hydroxy-2-cyclopentyl-2-phenyl acetamide,
(1α,5α,6α)-N-[3-benzyl-3-azabicyclo[3.1.0]hexyl-6-(aminomethyl)-yl]-2-hydroxy-2,2-diphenyl acetamide,
3-azabicyclo[3.1.0]hex-3-yl]but-2-ynyl-2-cyclopentyl-2-hydroxyphenyl acetate,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide,
(1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide, and
(1α,5α,6α)-6-N-(3-azabicyclo[3.1.0]hexyl-3-(4-methyl-3-pentenyl))-2-cyclopentyl-2-hydroxy-2-phenyl acetamide, and
their pharmaceutically acceptable salts, pharmaceutically acceptable solvates, enantiomers, diastereomers, polymorphs, N-oxide or metabolites.

8. The pharmaceutical composition according to claim 6 wherein the bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide L-(+)-tartrate salt,
(1α,5α,6α)-[3-benzyl-3-azabicyclo[3.1.0}hexyl-6-(methyl)-yl]-2-hydroxy-2,2-diphenyl acetate L(+)-tartrate salt,
(1α,5α,6α)-[3-benzyl-3-azabicyclo[3.1.0]hexyl-6-(methyl)-yl]-2-hydroxy-2-cyclohexyl-2-phenyl acetate L(+)-tartrate salt,
(1α,5α,6α)-[3-benzyl-3-azabicyclo[3.1.0]-hexyl-6-(methyl)-yl]-2-hydroxy-2-cyclopentyl-2-phenyl acetate L(+)-tartrate salt,
(2R)-(+)-(1α,5α,6α)-N-[3-benzyl-3-azabicyclo[3.1.0]hexyl-6-(aminomethyl)-yl]-2-hydroxy-2-cyclopentyl-2-phenyl acetamide L(+)-tartrate salt,
(2R,2S) (1α,5α,6α)-N-[3-azabicyclo[3.1.0]hexyl-6-(aminomethyl)-yl]-2-hydroxy-2-cyclopentyl-2-phenyl acetamide hydrochloride salt,
(2R)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2S)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2R,2S) (1α,5α,6α)-N-[3-azabicyclo[3.1.0]hexyl-6-(aminomethyl)-yl]-2-hydroxy-2-(3,3-difluorocyclopentyl)-2-phenyl acetamide tartrate salt,
(2R,2S) (1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamide tartrate salt,
(2R,2S)-N-[(1α,5α,6α)-3-azabicyclo[3.1.0]-hex-6-ylmethyl]-2-isopropyl-2-hydroxy-2-phenyl acetamide hydrochloride salt,
N-{[(1α,5α,6α)-3-chloro-3-azabicyclo[3.1.0]hex-6ylmethyl]}-2-cyclopentyl-2-hydroxy-2-phenyl acetamide hydrochloride salt,
(2R)-(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide tartrate salt,
(2R)-(1α,5α,6α)-N-[3-benzyl-3-azabicyclo[3.1.0]hexyl-6-(aminomethyl)-yl]-2-[(1S or 1R)-3,3-difluorocyclopentyl]-2-hydroxy-2-phenyl acetamide tartrate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide succinate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide tartrate salt,
(2R,2S)-(1α,5α,6α)-N-[3-(1-phenylethyl)-3-azabicyclo[3.1.0]hexyl-6-(amino)-yl]-2-hydroxy-2-cyclopentyl-2-phenyl acetamide tartrate salt,
(2R)-(1α,5α,6α)-N-[3-benzyl-3-azabicyclo[3.1.0]hexyl-6-(aminomethyl)-yl]-2-hydroxy-2-cyclopentyl-2-phenyl acetamide tartrate salt,
(1α,5α,6α)-N-[3-benzyl-3-azabicyclo[3.1.0]hexyl-6-(aminomethyl)-yl]-2-hydroxy-2,2-diphenyl acetamide tartrate salt,
2R(+),4[(1R,5S)-3-azabicyclo[3.1.0]hex-3-yl]but-2-ynyl-2-cyclopentyl-2-hydroxyphenyl acetate hydrochloride,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide L(+) tartrate salt,
(2R) (1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(2R)-(1α,5α,6α)-6-N-(3-azabicyclo[3.1.0]hexyl-3-(4-methyl-3-pentenyl))-2-cyclopentyl-2-hydroxy-2-phenyl acetamide succinate salt,
(2R)-(1α,5α,6α)-6-N-(3-azabicyclo[3.1.0]hexyl-3-(4-methyl-3-pentenyl))-2-cyclopentyl-2-hydroxy-2-phenyl acetamide L(+) tartrate salt,
(1S)-(3R)-1-azabicyclo[2,2,2]oct-3-yl-3,4-dihydro-1-phenyl-2(1H)-isoquinoline carboxylate,
(1S)-(3R)-1-azabicyclo[2,2,2]oct-3-yl-3,4-dihydro-1-phenyl-2(1H)-isoquinoline carboxylate succinate salt,
2-methyl propanoic acid 2-[(1R)-3-[bis(1-methylethyl)amino]-1-phenylpropyl]-4-(hydroxymethyl)phenyl ester and
2-methyl propanoic acid 2-[(1R)-3-[bis(1-methylethyl)amino]-1-phenylpropyl]-4-(hydroxymethyl)phenyl ester with (2E)-2-butenedioate.

9. The pharmaceutical composition according to claim 1 wherein said 5α-reductase inhibitor is a type 1 or a type 2 or both a type 1 and type 2 or a dual type 1 and type 2 inhibitor.

10. The pharmaceutical composition according to claim 9 wherein the 5α-reductase inhibitor is a dual type 1 and type 2 inhibitor.

11. The pharmaceutical composition according to claim 10 wherein the dual type 1 and type 2 inhibitor is dutasteride.

12. The pharmaceutical composition according to claim 9 wherein the 5α-reductase inhibitor is a type 2 inhibitor.

13. The pharmaceutical composition according to claim 12 wherein the type 2 inhibitor is finasteride.

14. A pharmaceutical product or medicament comprising a first pharmaceutical composition of a tailored α₁ adrenoceptor antagonist, a second pharmaceutical composition of a bladder selective antagonist and optionally included a third pharmaceutical composition of 5α-reductase inhibitor.

15. A pharmaceutical product or medicament of claim 14 wherein the product or medicament is a combined preparation.

16. A pharmaceutical product or medicament according to claim 13 wherein the combined preparation is single dosage form.

17. A pharmaceutical product or medicament according to claim 13 wherein the combined preparation comprises separate dosage forms.

18. A pharmaceutical product or medicament according to claim 14 wherein the tailored α₁ AR antagonist is selective for α₁ₐ over α_{1b} subtype but non-selective for α₁ₐ over α_{1d} subtype.

19. A pharmaceutical product or medicament according to claim 14 wherein the tailored α₁ AR antagonist is more than about 10 fold selective for α₁ₐ as compared to α_{1b} subtype and is less than about 10 fold selective for α₁ₐ over α_{1d} subtype in receptor binding and in vitro functional assay.

20. The pharmaceutical product or medicament according to claim 19 wherein the tailored α₁ adrenoceptor antagonist is selected from:
1-{3-[4-(2-methoxyphenyl) piperazin-1-yl]-propyl}-piperidine-2,6-dione,
2-[3-{4-(2-isopropoxyphenyl)piperazin-1-yl}propyl]-3a,4,7,7a-tetrahydro-1H-isoindole-1,3(2H)-dione,
5-[2-[[2-(2-ethoxyphenoxy)ethyl]amino]propyl]-2-hydroxybenzenesulfonamide,
and their pharmaceutically acceptable salts, pharmaceutically acceptable solvates, enantiomer, racemate, polymorphs, N-oxides or metabolites.

21. The pharmaceutical product or medicament according to claim 19 wherein the tailored α₁ adrenoceptor antagonist is selected from:
1-{3-[4-(2-methoxyphenyl) piperazin-1-yl]-propyl}-pipeiidine-2,6-dione hydrochloride salt,
2-[3-{4-(2-isopropoxyphenyl)piperazin-1-yl}propyl]-3a,4,7,7a-tetrahydro-1H-isoindole-1,3(2H)-dione hydrochloride salt and
5-[2-[[2-(2-ethoxyphenoxy)ethyl]amino]propyl]-2-hydroxybenzenesulfonamide hydrochloride salt,

22. A pharmaceutical product or medicament according to claim 14 wherein the bladder-selective antagonist is an agent which exhibits greater potency in inhibiting the carbachol-induced response on the bladder than the carbachol-evoked salivation when evaluated simultaneously in in vivo model in rabbit or dog.

23. A pharmaceutical product or medicament according to claim 19 wherein the bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide,
(1α,5α,6α)-[3-benzyl-3-azabicyclo[3.1.0}hexyl-6-(methyl)-yl]-2-hydroxy-2,2-diphenyl acetate,
(1α,5α,6α)-[3-benzyl-3-azabicyclo[3.1.0]hexyl-6-(methyl)-yl]-2-hydroxy-2-cyclohexyl-2-phenyl acetate,
(1α,5α,6α)-[3-benzyl-3-azabicyclo[3.1.0]-hexyl-6-(methyl)-yl]-2-hydroxy-2-cyclopentyl-2-phenyl acetate,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamnide,
N-[(1α,5α,6α)-3-azabicyclo[3.1.0]-hex-6-ylmethyl]-2-isopropyl-2-hydroxy-2-phenyl acetamide,
N-{[(1α,5α,6α)-3-chloro-3-azabicyclo[3.1.0]hex-6ylmethyl]}-2-cyclopentyl-2-hydroxy-2-phenyl acetamide,
(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide,
(1α,5α,6α)-N-[3-benzyl-3-azabicyclo[3.1.0]hexyl-6-(aminomethyl)-yl]-2-hydroxy-2-cyclohexyl-2-phenyl acetamide,
(1α,5α,6α)-N-[3-(1-phenylethyl)-3-azabicyclo[3.1.0]hexyl-6-(amino)-yl]-2-hydroxy-2-cyclopentyl-2-phenyl acetamide,
(1α,5α,6α)-N-[3-benzyl-3-azabicyclo[3.1.0]hexyl-6-(aminomethyl)-yl]-2-hydroxy-2,2-diphenyl acetamide,
3-azabicyclo[3.1.0]hex-3-yl]but-2-ynyl-2-cyclopentyl-2-hydroxyphenyl acetate,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide,
(1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide,
and their pharmaceutically acceptable salts, pharmaceutically acceptable solvates, enantiomer, racemate, polymorphs, N-oxides or metabolites.

24. A pharmaceutical product or medicament according to claim 22 the wherein bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide L-(+)-tartrate salt,
(1α,5α,6α)-[3-benzyl-3-azabicyclo[3.1.0}hexyl-6-(methyl)-yl]-2-hydroxy-2,2-diphenyl acetate L(+)-tartrate salt,
(1α,5α,6α)-[3-benzyl-3-azabicyclo[3.1.0]hexyl-6-(methyl)-yl]-2-hydroxy-2-cyclohexyl-2-phenyl acetate L(+)-tartrate salt,
(1α,5α,6α)-[3-benzyl-3-azabicyclo[3.1.0]-hexyl-6-(methyl)-yl]-2-hydroxy-2-cyclopentyl-2-phenyl acetate L(+)-tartrate salt,
(2R)-(+)-(1α,5α,6α)-N-[3-benzyl-3-azabicyclo[3.1.0]hexyl-6-(aminomethyl)-yl]-2-hydroxy-2-cyclopentyl-2-phenyl acetamide L(+)-tartrate salt,
(2R,2S) (1α,5α,6α)-N-[3-azabicyclo[3.1.0]hexyl-6-(aminomethyl)-yl]-2-hydroxy-2-cyclopentyl-2-phenyl acetamide hydrochloride salt,
(2R)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2S)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2R,2S) (1α,5α,6α)-N-[3-azabicyclo[3.1.0]hexyl-6-(aminomethyl)-yl]-2-hydroxy-2-(3,3-difluorocyclopentyl)-2-phenyl acetamide tartrate salt,
(2R,2S) (1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamide tartrate salt,
(2R,2S)-N-[(1α,5α,6α)-3-azabicyclo[3.1.0]-hex-6-ylmethyl]-2-isopropyl-2-hydroxy-2-phenyl acetamide hydrochloride salt,
N-{[(1α,5α,6α)-3-chloro-3-azabicyclo[3.1.0]hex-6ylmethyl]}-2-cyclopentyl-2-hydroxy-2-phenyl acetamide hydrochloride salt,
(2R)-(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide tartrate salt,
(2R)-(1α,5α,6α)-N-[3-benzyl-3-azabicyclo[3.1.0]hexyl-6-(aminomethyl)-yl]-2-[(1S or 1R)-3,3-difluorocyclopentyl]-2-hydroxy-2-phenyl acetamide tartrate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide succinate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide tartrate salt,
(2R,2S)-(1α,5α,6α)-N-[3-(1-phenylethyl)-3-azabicyclo[3.1.0]hexyl-6-(amino)-yl]-2-hydroxy-2-cyclopentyl-2-phenyl acetamide tartrate salt,
(2R)-(1α,5α,6α)-N-[3-benzyl-3-azabicyclo[3.1.0]hexyl-6-(aminomethyl)-yl]-2-hydroxy-2-cyclopentyl-2-phenyl acetamide tartrate salt,
(1α,5α,6α)-N-[3-benzyl-3-azabicyclo[3.1.0]hexyl-6-(aminomethyl)-yl]-2-hydroxy-2,2-diphenyl acetamide tartrate salt,
2R(+),4[(1R,5S)-3-azabicyclo[3.1.0]hex-3-yl]but-2-ynyl-2-cyclopentyl-2-hydroxyphenyl acetate hydrochloride,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide L(+) tartrate salt,
(2R) (1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(2R)-(1α,5α,6α)-6-N-(3-azabicyclo[3.1.0]hexyl-3-(4-methyl-3-pentenyl))-2-cyclopentyl-2-hydroxy-2-phenyl acetamide succinate salt,
(2R)-(1α,5α,6α)-6-N-(3-azabicyclo[3.1.0]hexyl-3-(4-methyl-3-pentenyl))-2-cyclopentyl-2-hydroxy-2-phenyl acetamide L(+) tartrate salt,
(1S)-(3R)-1-azabicyclo[2,2,2]oct-3-yl-3,4-dihydro-1-phenyl-2(1H)-isoquinolinecarboxylate,
(1S)-(3R)-1-azabicyclo[2,2,2]oct-3-yl-3,4-dihydro-1-phenyl-2(1H)-isoquinolinecarboxylate succinate salt,
2-methyl propanoic acid 2-[(1R)-3-[bis(1-methylethyl)amino]-1-phenylpropyl]-4-(hydroxymethyl)phenyl ester and
2-methyl propanoic acid 2-[(1R)-3-[bis(1-methylethyl)amino]-1-phenylpropyl]-4-(hydroxymethyl)phenyl ester with (2E)-2-butenedioate

25. A pharmaceutical product or medicament according to claim 14 wherein the 5α-reductase inhibitor is a type 1 or a type 2 or both a type 1 and type 2 or a dual type 1 and type 2 inhibitor.

26. A pharmaceutical product or medicament according to claim 25 wherein the 5α-reductase inhibitor is a dual type 1 and type 2 inhibitor.

27. A pharmaceutical product or medicament according to claim 26 wherein the dual type 1 and type 2 inhibitor is dutasteride.

28. A pharmaceutical product or medicament according to claim 25 wherein the 5α-reductase inhibitor is a type 2 inhibitor.

29. A product or medicament according to claim 28 wherein the type 2 inhibitor is finasteride.

30. method for treatment of a mammal suffering from lower urinary tract symptoms (LUTS) associated with or without BPH, comprising administering to said mammal, a therapeutically effective amount of a product or medicament, comprising a tailored α₁ AR antagonist, a bladder-selective antagonist and optionally included 5α-reductase inhibitor.

31. The method according to claim 30 wherein mammal is animal.

32. The method according to claim 30 wherein mammal is human.

33. The method according to claim 32 wherein human is man.

34. The method according to claim 32 wherein human is woman.

35. The method according to claim 30 wherein the said product or medicament is administered as a combined preparation.

36. The method according to claim 35 wherein the combined preparation is administered as single dosage form.

37. The method according to claim 35 wherein the combined preparation is administered in separate dosage forms.

38. The method according to claim 37 wherein the separate dosage forms are administered simultaneously.

39. The method according to claim 37 wherein the separate dosage forms are administered separately.

40. The method according to claim 37 wherein the separate dosage forms are administered sequentially.

41. The method according to claim 30 wherein the tailored α₁ AR antagonist is selective for α₁ₐ over α_{1b} subtype but non-selective for α₁ₐ over α_{1d} subtype AR antagonist.

42. The method according to claim 30 wherein the tailored α₁ AR antagonist is more than about 10 fold selective for α₁ₐ as compared to α_{1b} subtype and is less than about 10 fold selective for α₁ₐ as compared to α_{1d} subtype in receptor binding and functional assay.

43. The method according to claim 28 wherein the tailored α₁ AR antagonist is selected from:
1-{3-[4-(2-methoxyphenyl) piperazin-1-yl]-propyl}-piperidine-2,6-dione,
2-[3-{4-(2-isopropoxyphenyl)piperazin-1-yl}propyl]-3a,4,7,7a-tetrahydro-1H-isoindole-1,3(2H)-dione,
5-[2-[[2-(2-ethoxyphenoxy)ethyl]amino]propyl]-2-hydroxybenzenesulfonamide,
and their pharmaceutically acceptable salts, pharmaceutically acceptable solvates, enantiomer, racemate, polymorphs, N-oxides or metabolites.

44. The method according to claim 42 wherein the tailored α₁ AR antagonist is selected from:
1-{3-[4-(2-methoxyphenyl) piperazin-1-yl]-propyl}-pipeiidine-2,6-dione hydrochloride salt,
2-[3-{4-(2-isopropoxyphenyl)piperazin-1-yl}propyl]-3a,4,7,7a-tetrahydro-1H-isoindole-1,3(2H)-dione hydrochloride salt and
5-[2-[[2-(2-ethoxyphenoxy)ethyl]amino]propyl]-2-hydroxybenzenesulfonamide hydrochloride salt,

45. The method according to claim 30 wherein the bladder-selective antagonist is an agent which exhibits greater potency in inhibiting the carbachol-induced response on the bladder than the carbachol-evoked salivation when evaluated simultaneously in in vivo model in rabbit or dog.

46. The method according to claim 45 wherein the bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide,
(1α,5α,6α)-[3-benzyl-3-azabicyclo[3.1.0}hexyl-6-(methyl)-yl]-2-hydroxy-2,2-diphenyl acetate,
(1α,5α,6α)-[3-benzyl-3-azabicyclo[3.1.0]hexyl-6-(methyl)-yl]-2-hydroxy-2-cyclohexyl-2-phenyl acetate,
(1α,5α,6α)-[3-benzyl-3-azabicyclo[3.1.0]-hexyl-6-(methyl)-yl]-2-hydroxy-2-cyclopentyl-2-phenyl acetate,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2-cyclopentyl-2-phenyl acetamide,
(1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamnide,
N-[(1α,5α,6α)-3-azabicyclo[3.1.0]-hex-6-ylmethyl]-2-isopropyl-2-hydroxy-2-phenyl acetamide,
N-{[(1α,5α,6α)-3-chloro-3-azabicyclo[3.1.0]hex-6ylmethyl]}-2-cyclopentyl-2-hydroxy-2-phenyl acetamide,
(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide,
(1α,5α,6α)-N-[3-benzyl-3-azabicyclo[3.1.0]hexyl-6-(aminomethyl)-yl]-2-hydroxy-2-cyclohexyl-2-phenyl acetamide,
(1α,5α,6α)-N-[3-(1-phenylethyl)-3-azabicyclo[3.1.0]hexyl-6-(amino)-yl]-2-hydroxy-2-cyclopentyl-2-phenyl acetamide,
(1α,5α,6α)-N-[3-benzyl-3-azabicyclo[3.1.0]hexyl-6-(aminomethyl)-yl]-2-hydroxy-2,2-diphenyl acetamide,
3-azabicyclo[3.1.0]hex-3-yl]but-2-ynyl-2-cyclopentyl-2-hydroxyphenyl acetate,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide,
(1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(1α,5α,6α)-6-N-(3-azabicyclo[3.1.0]hexyl-3-(4-methyl-3-pentenyl))-2-cyclopentyl-2-hydroxy-2-phenyl acetamide, and
(1α,5α,6α)-6-N-(3-azabicyclo[3.1.0]hexyl-3-(4-methyl-3-pentenyl))-2-cyclopentyl-2-hydroxy-2-phenyl acetamide, and
their pharmaceutically acceptable salts, pharmaceutically acceptable solvates, enantiomers, diastereomers, polymorphs, N-oxide or metabolites.

47. The method according to claim 45 wherein the bladder-selective antagonist is selected from:
(1α,5α,6α)-N-[3-benzyl-3-azabicyclo[3.1.0]hexyl-6-(aminomethyl)-yl]-2-hydroxy-2-cyclopentyl-2-phenyl acetamide L-(+)-tartrate salt,
(1α,5α,6α)-[3-benzyl-3-azabicyclo[3.1.0}hexyl-6-(methyl)-yl]-2-hydroxy-2,2-diphenyl acetate L(+)-tartrate salt,
(1α,5α,6α)-[3-benzyl-3-azabicyclo[3.1.0]hexyl-6-(methyl)-yl]-2-hydroxy-2-cyclohexyl-2-phenyl acetate L(+)-tartrate salt,
(1α,5α,6α)-[3-benzyl-3-azabicyclo[3.1.0]-hexyl-6-(methyl)-yl]-2-hydroxy-2-cyclopentyl-2-phenyl acetate L(+)-tartrate salt,
(2R)-(+)-(1α,5α,6α)-N-[3-benzyl-3-azabicyclo[3.1.0]hexyl-6-(aminomethyl)-yl]-2-hydroxy-2-cyclopentyl-2-phenyl acetamide L(+)-tartrate salt,
(2R,2S) (1α,5α,6α)-N-[3-azabicyclo[3.1.0]hexyl-6-(aminomethyl)-yl]-2-hydroxy-2-cyclopentyl-2-phenyl acetamide hydrochloride salt,
(2R)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2S)-(1α,5α,6α)-N-[3-azabicyclo[3.1.0]hexyl-6-(aminomethyl)-yl]-2-hydroxy-2-cyclopentyl-2-phenyl acetamide hydrochloride salt,
(2R,2S) (1α,5α,6α)-N-[3-azabicyclo[3.1.0]hexyl-6-(aminomethyl)-yl]-2-hydroxy-2-(3,3-difluorocyclopentyl)-2-phenyl acetamide tartrate salt,
(2R,2S) (1α,5α,6α)-N-[3-azabicyclo[3.1.0]hexyl-6-(aminomethyl)-yl]-2-hydroxy-2,2-diphenyl acetamide,
N-[(1α,5α,6α)-3-azabicyclo[3.1.0]hex-6-ylmethyl]-2-phenyl-2-hydroxy-2-(N-methyl) phenyl acetamide tartrate salt,
(2R,2S)-N-[(1α,5α,6α)-3-azabicyclo[3.1.0]-hex-6-ylmethyl]-2-isopropyl-2-hydroxy-2-phenyl acetamide hydrochloride salt,
N-{[(1α,5α,6α)-3-chloro-3-azabicyclo[3.1.0]hex-6ylmethyl]}-2-cyclopentyl-2-hydroxy-2-phenyl acetamide hydrochloride salt,
(2R)-(1α,5α,6α)-N-[3-benzyl-3-azabicyclo[3.1.0]hexyl-6-(aminomethyl)-yl]-2-[(1R or 1S)-3,3-difluorocyclopentyl]-2-hydroxy-2-phenyl acetamide tartrate salt,
(2R)-(1α,5α,6α)-N-[3-benzyl-3-azabicyclo[3.1.0]hexyl-6-(aminomethyl)-yl]-2-[(1S or 1R)-3,3-difluorocyclopentyl]-2-hydroxy-2-phenyl acetamide tartrate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide succinate salt,
(2R,2S)-(1α,5α,6α)-N-[3-benzyl-3-azabicyclo[3.1.0]hexyl-6-(aminomethyl)-yl]-2-hydroxy-2-cyclohexyl-2-phenyl acetamide tartrate salt,
(2R,2S)-(1α,5α,6α)-N-[3-(1-phenylethyl)-3-azabicyclo[3.1.0]hexyl-6-(amino)-yl]-2-hydroxy-2-cyclopentyl-2-phenyl acetamide tartrate salt,
(2R)-(1α,5α,6α)-N-[3-benzyl-3-azabicyclo[3.1.0]hexyl-6-(aminomethyl)-yl]-2-hydroxy-2-cyclopentyl-2-phenyl acetamide tartrate salt,
(1α,5α,6α)-N-[3-benzyl-3-azabicyclo[3.1.0]hexyl-6-(aminomethyl)-yl]-2-hydroxy-2,2-diphenyl acetamide tartrate salt,
2R(+),4[(1R,5S)-3-azabicyclo[3.1.0]hex-3-yl]but-2-ynyl-2-cyclopentyl-2-hydroxyphenyl acetate hydrochloride,
N-methyl-N-(1α,5α,6α)-N-[3-(4-methyl-3-pentenyl)-3-azabicylo[3.1.0]-hex-6-yl]-2-cyclopentyl-2-hydroxy-2-phenyl acetamide L(+) tartrate salt,
(2R) (1α,5α,6α)-6-N-(3-azabicyclo[3.1.0]hexyl-3-(3,4-methylenedioxyphenyl)ethyl)-2-cyclopentyl-2-hydroxy-2-phenyl acetamide,
(2R)-(1α,5α,6α)-6-N-(3-azabicyclo[3.1.0]hexyl-3-(4-methyl-3-pentenyl))-2-cyclopentyl-2-hydroxy-2-phenyl acetamide succinate salt,
(2R)-(1α,5α,6α)-6-N-(3-azabicyclo[3.1.0]hexyl-3-(4-methyl-3-pentenyl))-2-cyclopentyl-2-hydroxy-2-phenyl acetamide L(+) tartrate salt,
(1S)-(3R)-1-azabicyclo[2,2,2]oct-3-yl-3,4-dihydro-1-phenyl-2(1H)-isoquinolinecarboxylate,
(1S)-(3R)-1-azabicyclo[2,2,2]oct-3-yl-3,4-dihydro-1-phenyl-2(1H)-isoquinolinecarboxylate succinate salt,
2-methyl propanoic acid 2-[(1R)-3-[bis(1-methylethyl)amino]-1-phenylpropyl]-4-(hydroxymethyl)phenyl ester and
2-methyl propanoic acid 2-[(1R)-3-[bis(1-methylethyl)amino]-1-phenylpropyl]-4-(hydroxymethyl)phenyl ester with (2E)-2-butenedioate

48. The method according to claim 30 wherein the 5α-reductase inhibitor is a type 1 or a type 2 or both a type 1 and type 2 or a dual type 1 and type 2 inhibitor.

49. The method according to claim 48 wherein the 5α-reductase inhibitor is a dual type 1 and type 2 inhibitor.

50. The method according to claim 49 wherein the dual type 1 and type 2 inhibitor is dustasteride.

51. The method according to claim 48 wherein the 5α-reductase inhibitor is a type 2 inhibitor.

52. The method according to claim 51 wherein the type 2 inhibitor is finasteride.
